(19) United States
(12) Patent Application Publication
(10) Pub. No.: US 2015/0374280 A1
(43) Pub. Date: Dec. 31, 2015

(54) BELT FOR GUIDING THE ACTIVATION OF THE CORE MUSCLES

(71) Applicant: CoRehab s.r.l., Trento (IT)

(72) Inventors: Roberto Tomasi, Ville di Giovo (IT); Dario Murgia, Ravina di Trento (IT); David Tacconi, Trento (IT)

(73) Assignee: CoRehab s.r.l., Trento (IT)

(21) Appl. No.: 14/766,374

(22) PCT Filed: Jan. 21, 2014
(86) PCT No.: PCT/EP2014/051509

§ 371 Date: Aug. 6, 2015

(30) Foreign Application Priority Data
Feb. 6, 2013 (IT) IT2013GE0016

(51) Int. Cl. A61B 5/00; A61B 5/11

(52) U.S. Cl. CPC A61B 5/486; A61B 5/1126; A61B 5/1107; A61B 5/6823; A61B 5/6831; A61B 5/4519; A61B 5/4571; A61B 5/6898; A61B 5/7455; A61B 5/7405; A61B 5/742; A61B 2562/0219; A61B 2562/0223; A61B 2503/10; A61B 2505/09; A61B 2560/0223; A61B 2562/164; A61B 2560/0475

(57) ABSTRACT

The invention is directed to a system and method using the system for guiding a person in correct activation of her or his core muscles for a sport or other exercise session The system is based on an elastic belt to be closely adhered to the lower abdomen of the human being and adapted to follow an inward movement of the lower abdominal wall without movements of the spine and pelvis effectively activating the core muscles responsible for low back control, sensor means provided at the belt for capturing extensions and contractions of the belt following said inward movement of the lower abdominal wall, means for evaluating the extensions and contractions of the belt following said lower abdominal wall region and for outputting evaluation data, means for comparing the evaluation data attained thereby to data representing correct activation of the core muscles, and feedback means provided with the belt for providing feedback to said result of said comparison to said person. In accordance with the invention the sensor means comprise fused together by a data fusion algorithm a 3D accelerometer sensor for capturing user's pelvis movements, a 3D gyroscope sensor for capturing user's pelvis movements, and/or a 3D Magnetometer sensor for capturing user's pelvis movements. The method evaluates and monitors based on the system said person's exercises by calculating the optimal position of the person in correct activation of her or his core muscles out of two positions taken up by the person in a calibration process.

The invention is directed to the field of controlling the position and movement of the central portion of a person's body, also called “Core stability”. More particularly, the invention provides a system for guiding a person in correct activation of his or her core muscles for a sport or other exercise session and a method for using the system.

In the following said person also is called a user of the method and both terms indicate the same human being.

### BACKGROUND OF THE INVENTION

It is the task of the core muscles to stabilize the spine of a human being. In healthy subjects, the core muscles activate immediately before any trunk or limb movements start to thereby protect the spine. In patients with back pain this activation of the core muscles however is significantly delayed. Although back pain settles spontaneously, core muscle function does not return spontaneously and re-training of the core muscles is needed to reduce recurrence of back pain.

The two core muscles that physiotherapists often focus on for assisting recovering of back pain patients are:
1. The Transverse Abdominis (TrA). This is the deepest layer of the abdominal muscles and when it contracts it pulls your navel in towards ther spine. This can be regarded as a kind of a body's natural corset, or even better, as a kind of a weightlifters belt that stabilizes the trunk. Research has shown that in moving subjects with healthy backs this muscle is activated on first before any other muscle, so that it is perfectly suited for safely stabilizing the spine. The transverse abdominis works together with the multifidus.
2. Multifidus is more like a small group of muscles that run from one vertebra in the lower back to the next one. These muscles are small and close to the spine and when they contract they work to stabilize each spinal segment. Particularly in a sporting session when the transverse abdominis are worked on the multifidus is worked on as well.

Retraining the deep muscular corset for recovering of back pain begins by motivating a patient to activate the core muscles one by one, usually starting with the transverse abdominis. This can be far more challenging than activating a muscle such as the biceps as the patient will often find it difficult to visualize the deep muscles, and there is no noticeable movement of the body involved.

Although exercises begin in static supine positions, the deep muscular corset muscles need to be retrained in all positions and movements including sitting, standing, driving, walking, miming and golfing for instance. It is important to activate these muscles several times throughout the day. Eventually, this conscious contraction will become an automatic response for the patient's body. This automatic contraction will then stabilize or anchor the spine in all movements, and protect the spine from re-injury.

It has been demonstrated (Richardson et al., “Therapeutic exercise for spinal segmental stabilization in low back pain: scientific basis and clinical approach”, London, Churchill Livingstone, 1999) that an inward movement of the lower abdominal wall without movements of the spine and pelvis effectively activates the TrA and multifidus responsible for low back control. Further research has shown that the inward movement of the lower abdominal wall activates the core muscles in a more effective way (for instance Urquhart et. al “Abdominal muscle recruitment during a range of voluntary exercises” Elsevier Manual Therapy, 10-2005, Behm at el “Trunk muscle electromyographic activity with unstable and unilateral exercises”, J Strength Cond Res. 2005 February; 19(1):193-201, Clark K M et al “Electromyographic comparison of the upper and lower rectus abdominis during abdominal exercises” J Strength Cond Res. 2003 August; 17(3):475-83).

### PRIOR ART

The WO 2009/013490 A1 discloses a system for guiding a person in correct activation of his core muscles for a sport or other exercise session of the kind defined by the features of the preamble of claim 1. In this known system the sensor means is based on a potentiometer, an optical sensor and a voltage meter thereby limiting the activation of the core muscles to their deformation.

The US 2011/0269601 A1 discloses a system and method for exercising core muscles, particularly the lumbar intrinsic musculature, including the multifidi. The system includes a first sensor for detecting upper body exertions of a user engaged in an exercise, a second sensor for detecting lower torso exertions for the user engaged in the exercise, a third sensor for detecting lower extremity exertions for the user engaged in the exercise, and a control system for processing sensor data from the first, second and third sensor. The control system includes a user interface for communicating information with the user, a data collection system for collecting sensor data, an analysis system for analyzing the sensor data and determining if the user is performing the exercise in a technically correct manner and a feedback system for alerting the user when the exercise is not being performed in the technically correct manner.

The EP 2231286 A2 discloses systems and methods for simultaneously contracting body core muscles and computerized instructional unit for facilitating same. The exercise apparatus also includes a vibration unit operable to cause all or portions of the exercise apparatus to vibrate.

The EP 2435142 A1 discloses a belt for training abdominal muscles and training method employing the same. The belt comprises means for determining a base girth of a user and means provided for determining changes in girth of the user as a result of contraction and relaxation of the user's abdominal muscles. Further means provide feedback to the user as to the extent of contraction of the user's abdominal muscles, the feedback being displayed as a continuous, progressive indication of the degree of contraction of the user's abdominal muscles. A training method employs the belt and comprises the steps of placing the belt around the waist of a user and determining a base girth of the user. The user's abdominal muscles are contracted and relaxed so as to provide feedback to the user as to the extent of contraction of the user's abdominal muscles, and a continuous, progressive indication of the degree of contraction of the user's abdominal muscles is noted.

The US 2005/0170938 A1 discloses a belt for feedback during abdominal core muscle exercise. This belt is provided with an inflatable bladder which, when inflated, is permitted to expand toward an interior of the belt and prevented by a barrier from expanding toward an exterior of the belt. A pressure gauge indicates the pressure within the bladder, and the gauge is fixedly displaced relative to the belt and the user such that the gauge may be viewed by a user when the belt is worn without significantly moving the cervical spine substantially out of a neutral posture.

The US 2012/0116259 A1 discloses a belt for training abdominal muscles comprises means for determining a base girth of a user. Means are provided for determining changes in girth of the user as a result of contraction and relaxation of the user's abdominal muscles. Further means provide feedback to the user as to the extent of contraction of the user's abdominal muscles, the feedback being displayed as a continuous, progressive indication of the degree of contraction of the user's abdominal muscles.

The U.S. Pat. No. 6,146,312 discloses a fabric belt for improving posture and abdominal muscle training. The belt includes a pair of segments formed of a non-elastic material coupled to an elastic material segment. The belt includes fabric attachment pads at its end portions to allow it to be secured to a wearer's torso. A sensor is secured across the elastic segment of the belt by a separate tension adjustment segment which is secured to one of the non-elastic segments by a second fabric attachment pad coupling. The sensor includes a motor and battery operatively coupled through a tension responsive switch. The motor rotates an off-center weight to produce a vibratory action when energized.

None of these known apparatus and methods is suited to assist a person in understanding the activation and maintenance of the core stability nor to assist her or him and/or an instructor in monitoring the core stability of his patients or athletes during any kind of workout, by alerting the person wearing the sensor on the correctness of the activation of the core muscles.

### DISCLOSURE OF THE INVENTION

An object underlying the invention is to provide a system for guiding a person in correct activation of his or her core muscles for a sport or other exercise session of the kind defined by the features of the preamble of claim 1 and a method for optimally using the system in order to assist said person in understanding the activation and maintenance of the core stability and/or to assist an instructor in monitoring the core stability of his patient or athlete during any kind of workout, and to alert the person wearing the sensor on the correctness of the activation of the core muscles.

Concerning the system this object is attained by the features of claim 1. Concerning the method this object is attained by the features of claim 6.

In contrast to the generic prior art defined by the WO 2009/013490 the invention provides for a coupling of known potentiometric measurements with measurements of accelerometers, gyroscopes and 3D magnetometers thereby effectivelyo assisting said person in understanding the activation and maintenance of the core stability and/or to assist an instructor in monitoring the core stability of his patient or athlete during any kind of workout, and to alert the person wearing the sensor on the correctness of the activation of the core muscles.

In particular, starting from a resting position a person moves the navel toward the spine at the maximum, reaching a position B. When the person maintains the abdominal muscles in a position C between these two positions, without moving the pelvis and breathing normally, it is ensured that the core muscles are optimally activated.

The sensor equipped elastic belt of the invention is adapted to measure this particular C position following a simple calibration through which it measures the positions A and B for calculating the C position therefrom.

The belt consists of elastic textile materials and includes resistive or capacitive sensors for measuring extensions and contractions of the material itself The belt needs to precisely adhered to the lower abdomen in order to capture introversion and extraversion in the navel region.

The accelerometers and/or gyroscopes are hidden within the belt in order to be positioned on the iliac crests and they are for measuring eventual movements of the pelvis which may cancel the activation of the core muscles.

The belt also may include vibration actuators to provide tactile feedback to a user of the belt, a microcontroller unit, a wireless transceiver, and a rechargeable battery.

By connecting the belt to a handheld device such as a smartphone or a tablet or to a personal computer, a software application is provided by the invention and adapted to guide the user through method steps for calibrating (measuring at positions A and B and calculating therefrom the position C) by means of the correct activation of the core muscles. The software also may be part of a circuit board integrated in the belt and supplied from a preferably re-chargeable battery. A simple audio-visual indicator in the software application indicates if the user is correctly holding the right position during a sport session, such as running, skiing, performing fitness and Pilates exercises or any other form of work-out. After a correct calibration through a visual interface, the vibration actuator of the invention provides for a tactile feedback to the user, indicating that he has to maintain in the correct position (position C). This feedback will be stopped as soon as the correct position is attained by the user.

The application uses a video indicator for indoor sessions, such as running on a treadmill, using gym machines or doing functional exercises. On the contrary, if the user is running carrying his smartphone, such hand-held device may indicate through audio feedbacks if the core muscles are still activated or not and which movements the user has to perform in order to reactivate these muscles correctly, even while running.

The application includes also a series of exercises designed to train the core stability. These exercises also need a strict control on the correct activation of the core muscles, so that the application will indicate whether or not the user is correctly training the core stability function.

The apparatus and device of the invention can be integrated into a rehabilitation system such as the one disclosed in the EP2510985 for improving the range and the quality of rehabilitation exercise that can be performed by measuring core stability functions.

### SHORT DESCRIPTION OF DRAWINGS

In the following preferred embodiments of the invention are described in detail along the enclosed drawings; in the drawings

FIG. 1 shows a user in positions corresponding to a correct activation of muscles;

FIG. 2 shows a user in positions corresponding to an incorrect activation of core muscles;

FIG. 3 shows a schematic diagram of a user and the device for guiding a user in correct activation of his core muscles of the invention;

FIG. 4 shows a sequence diagram of the user using the device of the invention;

FIG. 5 shows an embodiment of the belt of the device of the invention;

FIG. 6 shows an embodiment of the circuit diagram of the control unit of the device of the invention, and

FIG. 7 shows an embodiment of the flow chart of an algorithm used by the control unit of FIG. 6.

### DETAILED DESCRIPTION OF THE DRAWINGS

Our invention is composed of two main components: the sensor equipped belt and the software application for PC/smartphone/tablet/rehabilitation system interacting with the user as it can be seen in FIG. 3, where the whole system architecture is represented.

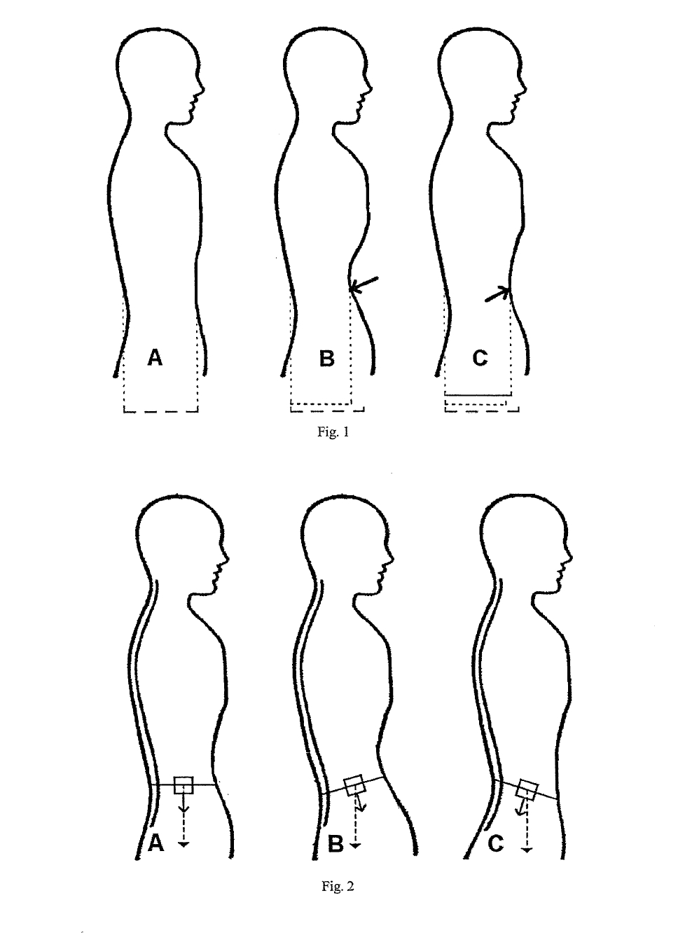

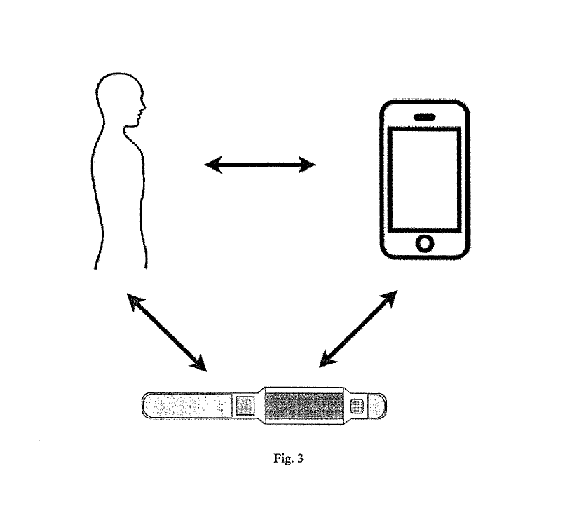

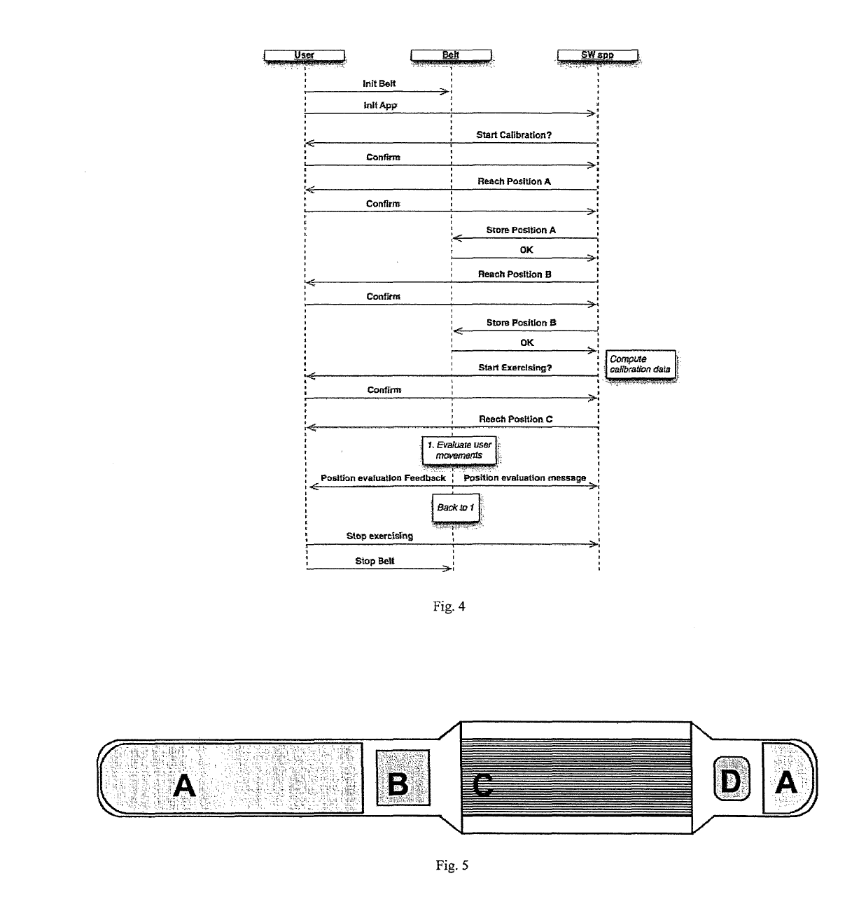

The present invention is in tracking and reporting the activation of core muscles of a subject for ensuring the correct activation of these muscles. The movement executed by a use in connection with said tracking and reporting is a right inward movement of the lower abdominal wall without movement of the spine and pelvis. The correct movement is represented in FIG. 1, where the user initially is in a rest position A. The user is performing a maximum inward movement of the lower abdomen to a position B and finally trying to reach a position C, in the middle between positions A and B and with a given percentage of the maximum inward movement. In FIG. 2 an incorrect activation of core muscles is represented, where the user is performing the right inward movement of the lower abdominal wall, while involving the back and pelvis. The movements of the user shown in FIGS. 1 and 2 are detected by the inertial sensors as shown in FIG. 2 (a box at a belt worn by the user represents inertial sensors and arrows depict the angle evaluated by this sensor during the user's movement), and the data thereby detected are combined with the data obtained by not shown strain sensors also positioned at the belt worn by the user in order to evaluate whether or not the core muscles are correctly activated.

Use of the belt in exercising which is shown in more detail in FIG. 5 is now described along FIG. 3.

FIG. 3. Shows a schematic diagram of a user and a hand-held device for guiding a user in correct activation of his core muscles of the invention based on a software application for controlling the user's movement which application is implemented in the hardware of a control circuit of the device.

As soon as the belt has been attached to the user's hip, after the initialization of the system, the user is guided by the application to carry out an initial calibration procedure by means of audio-visual indications provided on the hand-held device and parameters connected with the rest position ‘A’ and the final position ‘B’ are stored in a control circuit's memory of the device. The software application sends proper commands to the belt through a wireless connection, allowing for the storage of the signals output by the sensors in ‘A’ and ‘B’ positions after proper A/D conversion of those signals. These data are computed in the control circuit to extract and save control parameters into the circuit's memory that will be used to verify the correct activation of the core muscles during core stability exercises or a regular training session. When the calibration procedure was successful—after checking the coherence and the validity of the computed parameters—the belt sends to the application a corresponding message, waiting thereafter for user's confirmation to start training Otherwise the procedure is repeated providing for the user appropriate audio-visual indications until the user confirms to start training

When the user is ready to perform his exercises for training, he has to start the activation of the core muscles activity controlled by the software application sending the corresponding command to the belt. The microcontroller embedded into the belt as control circuit continuously samples the signals coming from the capacitive or resistive strain sensors and inertial sensors, for instance 3D accelerometers, gyroscopes and/or magnetometers. The microcontroller computes ‘on-the-fly’ the data obtained from these signals by A/D including proper filtering, whereafter these data are processed by data-fusion algorithms, for example by moving averages and using IIR/FIR filters, Kalman filters, Wiener-Kolmogorov filters, etc., for extracting suitable parameters to be compared with the parameters stored during the preceding calibration procedure. The final goal of this system is to verify that the user has taken up and is maintaining the C position with a certain amount of tolerance and thus correctly activating the core muscles to improve core stability.

After every processing of new data, the microcontroller stores the computed parameters in the memory embedded into the belt, and sends a message to the software application indicating whether the user takes up and maintains C position or not. As a consequence, the application provides audio-visual information from the hand-held device or the belt to the user indicating correct or incorrect position. If the position took up and maintained by the user is incorrect and hence the core muscles are not activated properly the microcontroller activates the vibration actuator embedded in the belt, providing al feedback to the user in order to motivate the user to take up and maintain the correct C position.

Audio-visual feedbacks provided by the software application and tactile feedbacks provided by the belt allow the user to verify each and every moment of activation of the enabling the user to recognize the core muscle activation as being correct or not and to correct his position in case of an incorrect muscle activation resulting in an effective improvement of core stability.

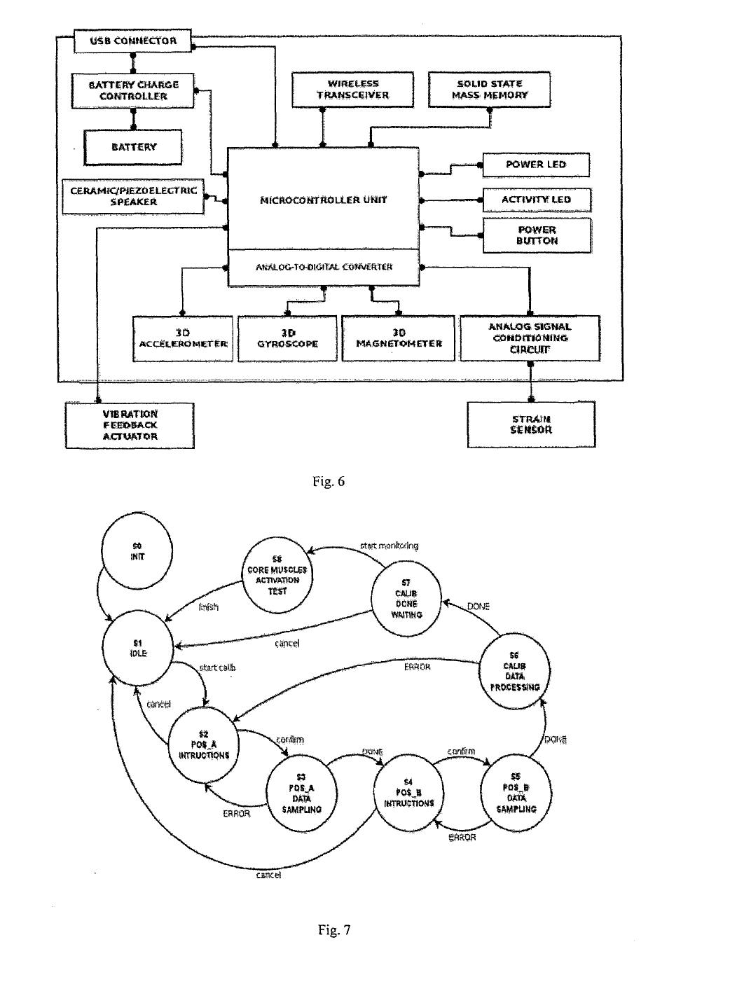

FIG. 5 shows an embodiment of the sensor equipped elastic belt of the device of the invention in more detail. The belt includes:
a strain sensor (C) positioned in correspondence to lower abdominal region when the elastic belt is put on the user's hip;
an electronic board (B), including the inertial sensors, the microcontroller, the wireless transceiver and the other components of the device shown in more detail in FIG. 6;
the vibration feedback actuator (D);
elastic textile portions, and
an inextensible portion corresponding to a belt coupling system (A).

The whole processing of the data received from the sensors is performed by the control circuit implemented as microcontroller incorporated in the elastic belt, both during the initial calibration process and the following position-monitoring process. Processing is performed through suitable algorithms optimized to be implemented on embedded devices. In this way the transfer of data sent to and received from the software application is chosen to be as low as possible, allowing for a longer lifespan of a battery providing the needed electric energy. A faster data exchange through wireless communication would result in a short battery lifespan not suited for ensuring core muscles monitoring during long training sessions or during outdoor activities.

The apparatus, device and method of the invention are based on a special client-server wireless communication algorithm minimizing the amount of data exchange and therefore maximizing the battery lifespan. In the algorithm the elastic belt is represents the client and the software application represents the server. In fact, the system reduces data exchange to commands sent from the server (i.e. the software application) to the client (i.e. the belt) and to messages (with data already processed) sent from client to server.

Furthermore, the complexity on the server side is drastically reduced, allowing an implementation of the software application on a wide range of devices, including relatively simple smartphones and tablets. At the end of the training session, the user can download the data from the solid-state mass memory of the elastic belt to the software application allowing for of analyzing these data to evaluate user's performance.

As shown in FIG. 6 the circuit diagram of the control unit of the device of the invention, the electronic or circuit board, which is embedded into the sensor equipped elastic belt is composed of the following components:
a rechargeable battery serving as the power source for the whole system (micro-controller, wireless transceiver and all the other peripherals);
a battery charge controller for limiting the rate at which electric current is supplied to or drawn from the battery, preventing overcharging, overvoltage and complete drain, all in favor lifespan and safety;
an USB connector for re-charging the battery, downloading data stored in the memory and updating microcontroller's firmware;
a wireless transceiver for creating a serial wireless link between the micro-controller and the software application, receiving commands from the application, sending back response messages during set-up and calibration processes and transmitting data processed by the microcontroller during the core muscles monitoring process;
a solid-state mass memory for storing calibration parameters sampled and processed during an initial set-up wizard, and logging of the core muscles activity during the monitoring process. All these data can be downloaded at the end of the training session to evaluate the quality of the performance;
a microcontroller serving as heart of the whole system and connected to all the peripherals of the electronic board. The microcontroller controls the battery charge and the re-charge process through the battery controller, receives and sends data from/to the server (where the front-end application is running) through the wireless transceiver, samples analog signals coming from all the sensors (accelerometers, gyroscopes, magnetometers, strain sensors) through the ADC (which may be embedded into the micro-controller or a stand-alone module), elaborates them with filtering and data-fusion algorithms, writes processed data to the solid-state mass memory, controls the power led and activity led status, activates the vibration actuator during the monitoring process in case of wrong position, and manages the stored data transfer from the solid-state mass memory to the server at the end of the exercises session;
a power led which is active only when the elastic belt is turned on;
an activity led blinking when the wireless connection with the host is established and provides feedback on battery status or other general information;
a power button for switching the device on off;
a 3D accelerometer sensor for capturing user's pelvis movements in conjunction with other inertial sensors;
a 3D gyroscope sensor for capturing user's pelvis movements in conjunction with other inertial sensors;
a 3D Magnetometer sensor for capturing user's pelvis movements in conjunction with other inertial sensors;
an analog signal conditioning circuit connected with the resistive or capacitive strain sensor for manipulating the analog signal received from the sensors by meeting the requirements of the ADC front-end. This circuit in accordance to the kind of the sensor (capacitive or resistive) may include a Wheatstone bridge, a charge sensitive preamplifier, a low-noise amplifier, an anti-alias filter, etc., and
a ceramic/piezoelectric loudspeaker providing audio feedback during the calibration procedure and during the core muscles monitoring process;

Further, the electronic board is connected with two other components embedded in the elastic belt:
strain sensors included in the elastic textile materials of the belt and adapted to measure extensions and contractions of the belt material itself for providing an analog signal correlated to the amount of stretching, and
a vibration actuator providing tactile feedback during the core muscles monitoring process in case of an incorrect position of the user detected by the processing algorithm of the micro-controller and displaying an incorrect muscle activation of the user.

In the following an embodiment of the microcontroller algorithm providing for the advantages described above is described in detail.

The microcontroller of the sensor equipped elastic belt is programmed with the algorithm which in conjunction with the software application is adapted to guide the user through the steps for calibrating and measuring a correct activation of the core muscles. The main steps of the method underlying this algorithm are depicted in the flow diagram of FIG. 7 and are as follows:

S0—Init:

This is the very first step of the algorithm which becomes loaded when the device is switched on. The microcontroller initializes all the peripherals and waits for an incoming connection from the server device through the wireless transceiver module. When a proper link is established with the software application the microcontroller jumps to the next step S1.

S1—Idle

Following to the initialization procedure, the microcontroller goes into idle mode, waiting for commands from the software application. If the received command is “start calibration” sent from the software application when the user wants to use the sensor equipped elastic belt the microcontroller jumps to the next step S2, otherwise it remains in the current step S1 waiting for proper command

S2—Position A Instructions

The microcontroller now sends a message to the server showing to the user the right way to take up the A position through audio and/or video instructions, and waiting for commands from the software application. When the user is ready and selects the “confirm” button on the server, a corresponding message is sent to the microcontroller then jumping to the next step S3. Otherwise, if the user doesn't want to continue and selects the “cancel” button on the application, a corresponding message is sent to the microcontroller then returning back to the step S1.

S3—Position A Data Sampling

In this step the microcontroller samples the signals from all the sensors for a certain amount of time and performs a first raw processing of the acquired data, to check if the user didn't move during calibration time. If the test has positive outcome, the microcontroller sends a “done” message to the application and jumps to the next step S4. Otherwise an “error” message is sent to the server and the microcontroller returns back to the step S2.

S4—Position B Instructions

In this step the microcontroller sends a message to the server showing to the user the right way to take up the B position through audio and/or video instructions, and waiting for commands from the software application. When the user is ready and selects the “confirm” button on the application a corresponding message is sent to the microcontroller which jumps to the next step S5. Otherwise, if the user doesn't want to continue and selects the “cancel” button on the application, a corresponding message is sent to the microcontroller which returns back to the step S1.

S5—Position B Data Sampling

In this step the microcontroller samples the signals from all the sensors for a certain amount of time and performs a first raw processing of the acquired data to check the user didn't move during calibration time. If the test has a positive outcome, the microcontroller sends a “done” message to the application and jumps to the next step S6, otherwise an “error” message is sent to the server and the microcontroller returns back to the step S4.

S6—Calibration Data Processing

This is the last step of the calibration procedure: the microcontroller processes the data acquired during steps S3 (position A) and S5 (position B) to test if they can be used to obtain proper parameters for the monitoring activity. If the test has a positive outcome, the microcontroller computes position C upper and lower parameters, saving them in the solid-state mass memory, sending out a “done” message to the application and jumping to the next step S7. Otherwise, an “error” message is sent to the server and the microcontroller returns back to the step S2.

S7—Calibration Done—Waiting for Start Monitoring

Following to the calibration procedure, the microcontroller goes into idle mode, waiting for commands from the software application. If the received command is “start monitoring” (sent from the software application when the user wants to monitor the core muscles activity during his core stability exercises) the microcontroller sends the corresponding message to the server and jumps to the next step S8. If the user however doesn't want to continue the exercises and selects the “cancel” button on the application, a corresponding message is sent to the microcontroller which then returns back to the step S1. Otherwise, it remains at the current step S7 waiting for proper command.

S8—Core Muscles Activation Test Loop

Now the microcontroller executes the core muscles activation test loop until a “stop” message is received from the application (in that case it returns to the step S1). At every iteration of the test loop, the microcontroller samples data from all the sensors and processes them “on-the-fly” with filtering and data-fusion algorithms to compute various parameters correlated to the core muscles activation. These algorithms are fed by the input coming from strain and inertial sensors and evaluate if a shortening of strain sensors happened without involving pelvis movement. After processing, the new parameters are saved to the solid-state mass memory and compared with the upper and lower position C parameters computed during calibration procedure. If the computed parameters are between the position C parameters, a “right position” message is sent to the application and the vibration actuator is turned off. Otherwise a “wrong position” message is sent to the application and the vibration actuator is activated to provide tactile feedback. The same happens if position C is reached but the microcontroller computes a movement of the pelvis region through the inertial sensors. The software application interface shows core muscles activation status to the user, according to the message received from the microcontroller.

Based on the above described method steps the algorithm pseudo-code (state machine) is as follows:

```
## S0 - Init
init_sensors( )
init_transceiver_module( )
configure_transceiver_module( )
next_state(S1)
##S1 - Idle
while loop
  send_message(WAITING_FOR_COMMAND)
  command = get_command( )
  if (command is_empty)
    pass
  else if (command == start_calib)
    next_state(S2)
  else
    send_message(WRONG_COMMAND)
## S2 - PosA Instructions
send_message(posA_INSTRUCTIONS)
clear_data(posA_data)
while loop
  command = get_command( )
  if (command is_empty)
    pass
  if (command == confirm)
    next_state(S3)
  else if (command == cancel)
    next_state (S1)
  else
    send_message(WRONG_COMMAND)
## S3 - PosA Data Sampling
posA_data = get_data(strain_sensor, accelerometers, gyroscopes, magnetometers)
test_data = check_data_integrity(posA_data)
if (test_data == pass)
  send_message(DONE)
  next_state(S4)
else if (test_data == fail)
  send_message(ERROR)
  next_state(S2)
## S4 - PosB Instructions
send_message(posB_INSTRUCTIONS)
clear_data(posB_data)
while loop
  command = get_command( )
  if (command is_empty)
    pass
  if (command == confirm)
    next_state(S5)
  else if (command == cancel)
    next_state(S1)
  else
    send_message(WRONG_COMMAND)
## S5 - PosB Data Sampling
posB_data = get_data(strain_sensor, accelerometers, gyroscopes, magnetometers)
test_data = check_data_integrity(posB_data)
if (test_data == pass)
  send_message(DONE)
  next_state(S6)
else if (test_data == fail)
  send_message(ERROR)
  next_state(S4)
##S6 - Calib Data Processing
test_parameters =check_parameters_coherence(posA_data, posB_data)
if (test_parameters == pass)
  posC_lower_limit_parameters = compute_calib_data(posA_data, posB_data)
  posC_upper_limit_parameters = compute_calib_data(posA_data, posB_data)
  send_message(DONE)
  next_state(S7)
else if (test_parameters == fail)
  send_message(ERROR)
  next_state(S2)
##S7 - Calib Done - Waiting for start monitoring
while loop
  send_message(WAITING_FOR_COMMAND)
  command = get_command( )
  if (command is_empty)
    pass
  else if (command == start_monitoring)
    next_state(S8)
  else if (command == cancel)
    next_state(S1)
  else
    send_message(WRONG_COMMAND)
##S8 - Core Muscles Activation test loop
while loop
  command = get_command( )
  if (command is_empty)
    new_data = get_data(strain_sensor, accelerometers, gyroscopes, magnetometers)
    new_parameters = SIFDA(new_data)
  if (posC_Jowerlimit_parameters < new_parameters < posC_upper_limit_parameters)
    send_message(RIGHT_POSITION)
    set_vibration_feedback(off)
  else
    send_message(WRONG_POSITION)
    set_vibration_feedback(on)
  else if (command == finish)
    next_state(S1)
  else
    send_message(WRONG_COMMAND)
##SIFDA(strain_sensor, accelerometers, gyroscopes, magnetometers)
inertial_data = kalman_filter(accelerometers, gyroscopes, magnetometers)
muscles_activity = compute_strain(strain_sensor)
parameters = data_fusion_algorithm(muscles_activity, inertial_data)
return parameters
```

The microcontroller also is responsible for executing the fusion of data coming from the various inertial and strain sensors through a so called Strain and Inertial Data Fusion Algorithm (SIDFA) which is based on the following method steps:

This method and the SIDFA which is based thereon is executed both during calibration of the sensor equipped elastic belt and during the core muscles monitoring activity. During calibration the microcontroller samples and stores the signals coming from the strain sensors with respect to positions A and B. In particular, the sampled signals are computed in order to evaluate a length measurement: the measurements corresponding to position A and position B it is referred to with La and Lb, respectively. Furthermore, during calibration, the microcontroller samples and stores the signals received from the inertial sensors, knowing in advance the position of the user during calibration (i.e. if the user is standing, sitting, supine, prone, etc . . . ). These data are converted and elaborated with a Kalman filter in order to obtain pitch, roll and yaw initial angles which are referred as Ø_{—}0p, Ø_{—}0r, Ø_{—}0y. Obviously, during calibration, the method and SIDFA check if the Ø angles have changed while moving from A to B, and in this case the calibration procedure needs to be restarted (as described in previous section).

For the following ∂L is defined as

∂L=La−Lb

as being a difference between La and Lb. It is recalled that the method and SIFDA control whether the user is moving correctly toward position C in order to activate core muscles. To do so the method and SIFDA evaluate the tolerance range for position C which is referred to as range_c. The lower limit of range_c is

L_{c—}c_{—}0=K_tol*(−∂L/2)

while the upper limit is

L_{—}c_{—}1=K_tol*(∂L/2)

where K_tol is between 0 and 1 and decided by the application. The center of range_c, position C is then:

Lc=Lb+∂L/2

The smaller is K_tol the shorter is the tolerance range range_C around position C and such constant value can depend on the training exercise as well as the exercising program difficulty.

Concerning the pitch, roll and yaw angles Øp_tol, Ør_tol, Øy_tol are defined as a predefined tolerance angle around the central value evaluated during calibration. This value strictly depends on the type of exercise the user wants to perform for training core stability. For instance, during a skying session, the tolerance values will be as high to admit all the values of the angles since the angles vary directly with the movement of the exercise, while during more static exercises the user needs to control also the pelvis and low back stability and the tolerance angles will be very narrow. The amplitude of the ranges range_Øp, range_Ør, range_Øy is then equal to two times the size of the tolerance angle.

At this point, a control loop can be executed and will be effectively initiated when the user starts an exercising session signaled through the software application. The frequency of the control loop can vary from 10 to 250 Hz depending on to the necessary precision in measuring data and to the type of training At the i_th iteration, the method as well as SIFDA acquires real-time data from strain and inertial sensors and evaluates the following parameters:
the i_th elongation with respect to target position C is ∂L_i=L_i−Lb−∂L/2, where L_i is the i_th value read from strain sensors and converted by the microncontroller
the i_th pitch angle with respect to the initial value Ø_{—}0p is ∂Ø_ip=Ø_ip−Ø_{—}0p, where Ø_ip is the i_th pitch angle value read from inertial sensors after Kalman filtering
the i_th roll angle with respect to the initial value Ø_{—}0r is ∂Ø_ir=Ø_ir−Ø_{—}0r, where Ø_ir is the i_th roll angle value read from inertial sensors after Kalman filtering
the i_th yaw angle with respect to the initial value Ø_{—}0y is ∂Ø_iy=Ø_iy−Ø_{—}0y, where Ø_iy is the i_th yaw angle value read from inertial sensors after Kalman filtering

When each evaluated value is within the corresponding tolerance range, this means that the user is correctly activating the core muscles. In particular, the following equation is evaluated:

ΔC_{—}i=(∂L_{—}i BETWEEN range_{—}C)&&(∂Ø_{—}ip BETWEEN rangeØ_{—}p)&&(∂Ø_{—}ir BETWEEN rangeØ_{—}r)&&(∂Ø_{—}iy BETWEEN rangeØ_{—}y)

wherein the operator BETWEEN answers 1 if a value is within a given range, 0 otherwise.

If ΔC_i=1, the position is correct, otherwise it is incorrect.

1. A system for guiding a person in correct activation of his core muscles for a sport or other exercise session, comprising
an at least partially elastic belt to be closely adhered to the lower abdomen of the human being and adapted to follow the a proper inward movement of the lower abdominal wall without movements of the spine and pelvis effectively activating core muscles responsible for low back control,
sensor means provided at the belt for capturing extensions and contractions of the belt following said inward movement of the lower abdominal wall,
means for evaluating the extensions and contractions of the belt following said the lower abdominal wall region and for outputting evaluation data,
means for comparing the evaluation data attained thereby to data representing correct activation of the core muscles, and
feedback means provided with the belt for providing feedback to said result of said comparison to said person,
characterized in that the sensor means comprise fused together by a data fusion algorithm:
a 3D accelerometer sensor for capturing user's pelvis movements,
a 3D gyroscope sensor for capturing user's pelvis movements, and/or
a 3D Magnetometer sensor for capturing user's pelvis movements.

2. The system of claim 1, wherein:
the evaluation means is integrated in the belt and/or implemented in a separate device, preferably in a hand-held device or a personal computer;
the evaluation means is implemented as software; and/or
the evaluation means comprise storage means for storing the data representing correct activation of the core muscles.

3. The system of claim 2, wherein the feedback means comprises actuator means provided with the belt for providing the person a tactile feedback.

4. The system of claim 3, comprising means for informing said person and/or a therapist or trainer of the person of correctly holding the right position during the sport or other exercise session.

5. The system of claim 10, wherein the sensor means comprise strain sensors included in the elastic textile materials of the belt and adapted to measure extensions and contractions of the belt material itself.

6. A method for guiding a person in correct activation of his core muscles for a sport or other exercise session using the system of claim 1, comprising the following muscles activation test loop steps based on the said user taking up a C position corresponding to correct activation of his core muscles:
sampling data from the sensor means to compute various parameters correlated to the core muscles activation;
saving the various parameters in the memory and comparing these parameters with upper and lower parameters of the C position stored in the memory,
wherein when the various parameters are between the upper and lower parameters of the C position, a “right position” message is sent out to said person by the feedback means, and
wherein otherwise a “wrong position” message is sent out to said person by the feedback means.

7. The method of claim 6, wherein the muscles activation test loop steps are preceded by a calibration process for evaluating the C position, the calibration process comprising the steps:
sending a message to show to the user the right way to take up an A position through audio and/and or video instructions (step S2);
sampling the signals from the sensor means for a certain amount of time and performing a first raw processing of the acquired data, to check if the user didn't move after taking up the A position (step S3);
sending a message to show to the user the right way to take up a B position through audio and/and or video instructions (step S4);
sampling the signals from the sensor means for a certain amount of time and performing a first raw processing of the acquired data, to check if the user didn't move after taking up the B position (step S5);
processing the data acquired during steps S3 (position A) and S5 (position B) to test if these data can be used to obtain proper parameters for the following muscles activation test loop steps (step S6),
wherein if the test has a positive outcome, upper and lower parameters of the C position are computed and saved in the memory, and
wherein otherwise, an error message is sent out to said person.

8. The method of claim 7, wherein a waiting for start step (step 7) is interposed between the calibration process and the muscles activation test loop steps, wherein when said person wants to monitor the core muscles activity during his core stability exercises he initiates sending out of a corresponding message to start and the muscles activation test loop steps, and wherein otherwise the waiting for start step (step 7) is maintained.

9. The method of claim 8, wherein the method is implemented as a microcontroller algorithm, said microcontroller being implemented in the evaluation means.

10. The system of claim 3, comprising means for informing said person and/or a therapist or trainer of the person of correctly holding the right position during the sport or other exercise session;
the information means comprising a visual, and/or an aural indictor; and/or the feedback means comprising a vibration actuator providing tactile feedback in case of an incorrect position of said person during sport or other exercise session.
